(19) United States
(12) Patent Application Publication
(10) Pub. No.: US 2019/0150963 A1
(43) Pub. Date: May 23, 2019

(54) DEVICES AND METHODS FOR RESPIRATORY AIRWAYS BLEEDING MANAGEMENT AND TEMPORARY OCCLUSION OF AIRWAYS

(71) Applicant: MONTEFIORE MEDICAL CENTER, Bronx, NY (US)

(72) Inventor: Ali Sadoughi, Scarsdale, NY (US)

(73) Assignee: MONTEFIORE MEDICAL CENTER, Bronx, NY (US)

(21) Appl. No.: 16/097,632

(22) PCT Filed: May 15, 2017
(86) PCT No.: PCT/US17/32598

§ 371 Date: Oct. 30, 2018

Related U.S. Application Data
(60) Provisional application No. 62/337,924, filed on May 18, 2016.

(51) Int. Cl. A61B 17/24; A61B 1/018; A61B 1/267; A61B 17/50; A61M 29/02

(52) U.S. Cl. CPC A61B 17/24; A61B 1/018; A61B 2017/0034; A61B 17/50; A61M 29/02; A61B 1/2676

(57) ABSTRACT

Catheters and methods for controlling bleeding in airways and for occlusion of airways are provided that allow a bronchoscope to be withdrawn from the airway while an inflated balloon catheter remains in position in the airway, for example at a site of airway bleeding.

### CROSS REFERENCE TO RELATED APPLICATIONS

This application claims the benefit of U.S. Provisional Application No. 62/337,924, filed on May 18, 2016, the contents of which are hereby incorporated by reference.

### BACKGROUND OF THE INVENTION

Airway bleeding is a serious clinical situation that needs to be controlled promptly. Respiratory airways bleeding is a common encounter in the field of pulmonology, thoracic surgery and critical care. It is also one of the most fearful complications of pulmonary interventions including trans-bronchial lung biopsy. Even though there are some regular techniques to prevent or stop bleeding in the airways, there is no definite solution to control excessive bleeding. Professionals in this field are in need of more reliable methods.

Current techniques to control active bleeding in the respiratory airways are limited and include continuous suction with the help of a bronchoscope to keep the airways clean and to prevent spilling of blood to neighborhood airways and potentially stop bleeding with a likelihood of local vessels and airways collapse after applying the negative pressure with suction. Another maneuver is rotating the patient toward the side of bleeding (the bleeding side down) in order to prevent spilling of blood to the contralateral lung and to maintain sufficient ventilation. Use of cold saline has also been suggested; however, it is less useful when the bleeding site is in the peripheral lung with limited visualization. These maneuvers can fail especially if the bleeding is brisk or if the operator is not a skillful bronchoscopist.

Another alternative is using available inflating balloon catheters, but their usage is limited due to the following reasons. For catheters that pass through a working channel of a bronchoscope, one is unable to use the bronchoscope after the insertion of the balloon, since by design the bronchoscope cannot be removed without removing the balloon. Thus, the clinician is unable to move on to the next steps of intervention, including cleaning the blood and clots from neighboring airways after the balloon is inflated. For catheters that pass along, and outside a bronchoscope, it is difficult and time consuming to insert the balloon to the site of bleeding, and especially in an emergency situation of active airway bleeding, it is inefficient. For insertion of this type of balloon catheters, the bronchoscope first needs to be removed outside of the airway. This maneuver is prohibited in case of active bleeding in the airways, since the operator may fail to pass the bronchoscope back into the site of bleeding when the view is blocked by blood.

Thus, there is a need for innovations to provide a solution to this life threatening problem and to enable clinicians to treat this major medical emergency with more confidence. At the same time, such innovations would give peace of mind to pulmonologists who plan for conventional trans-bronchial biopsy, knowing they have a better backup plan in case of uncontrolled bleeding. The present invention provides improved devices and procedures for controlling airway bleeding.

### SUMMARY OF THE INVENTION

The present invention provides catheters for insertion into an airway of a patient through a channel of a bronchoscope, the catheters comprising a distal end, which is inserted into a patient, the distal end comprising a balloon that can be inflated and deflated; a proximal end, which is the end closest to a physician or operater when the distal end is inserted into the patient, the proximal end comprising an adaptor used to inflate and deflate the balloon, wherein the adaptor is detachable from, and reattachable to, the catheter; and a middle section between the distal end and the proximal end; the middle section comprising a compressible part that can be clamped to occlude the catheter or a valve that can be closed to occlude the catheter; wherein the catheter is dimensioned and configured to be inserted through a channel of a bronchoscope.

The invention provides methods of controlling bleeding in the airway, the methods comprising inserting any of the catheters disclosed herein with a deflated balloon into the airway adjacent to a site of bleeding, wherein the catheter is inserted into the airway through a channel of a bronchoscope; inflating the balloon to apply pressure to the site of bleeding to thereby control bleeding in the airway; partially withdrawing the bronchoscope by sliding the bronchoscope over the catheter, keeping the catheter in place and the balloon inflated; when the bronchoscope is withdrawn past the compressible part of the catheter or past the valve in the middle part of the catheter, either clamping the compressible part of the catheter or closing the valve to occlude the catheter, thereby keeping the balloon inflated; detaching the adaptor from the proximal end of the catheter, and fully withdrawing the bronchoscope past the proximal end of the catheter; and, optionally, reinserting the bronchoscope into the airway.

The invention also provides methods of controlling bleeding in the airway, the methods comprising inserting any of the catheters disclosed herein with a deflated balloon into the airway adjacent to a site of bleeding, wherein the catheter is inserted into the airway through a channel of a bronchoscope; detaching the adaptor from the proximal end of the catheter; removing the bronchoscope by sliding the bronchoscope over the catheter, keeping the catheter in place; reattaching the adapter to the proximal end of the bronchoscope; inflating the balloon to apply pressure to the site of bleeding to thereby control bleeding in the airway and, optionally, reinserting the bronchoscope into the airway.

The invention further provides methods of retracting a foreign body from the airway, the methods comprising inserting any of the catheters disclosed herein with a deflated balloon into the airway adjacent to the foreign body, wherein the catheter is inserted into the airway through a channel of a bronchoscope; detaching the adaptor from the proximal end of the catheter; removing the bronchoscope by sliding the bronchoscope over the catheter, keeping the catheter in place; reinserting the bronchoscope into the airway to visually guide the distal end of the catheter past the foreign body; reattaching the adapter to the proximal end of the bronchoscope; inflating the balloon; and either using the inflated balloon to retract the foreign body as the catheter with the inflated balloon is withdrawn from the airway, or using the inflated balloon to prevent movement of the foreign body within the airway while a tool is inserted into the airway to grab and retract the foreign body.

### BRIEF DESCRIPTION OF THE DRAWINGS

FIG. 1. Example of an embodiment of the catheter of the present invention. Drawing not to scale. “1” shows a balloon at the distal end of the catheter. The balloon is fully inflated in this illustration. The balloon can be deflated to allow passage of the catheter through a channel of a bronchoscope. “2” shows the shaft of the catheter. “3” shows a compressible part of the catheter. “4” illustrates a detachable clamp that can be applied to the compressible part of the catheter. “5” shows a detachable adaptor with a two-way stop cock at the proximal end of the catheter.

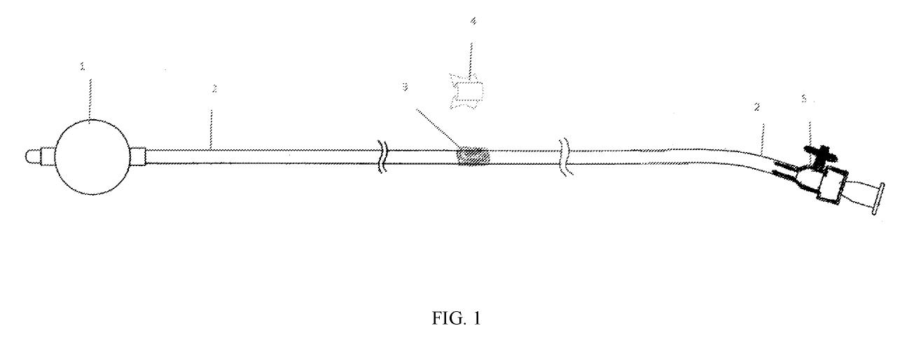

### DETAILED DESCRIPTION OF THE INVENTION

The present invention provides a catheter for insertion into an airway of a patient through a channel of a bronchoscope, the catheter comprising
a distal end, which is inserted into a patient, the distal end comprising a balloon that can be inflated and deflated;
a proximal end, which is the end closest to a physician or operater when the distal end is inserted into the patient, the proximal end comprising an adaptor used to inflate and deflate the balloon, wherein the adaptor is detachable from, and reattachable to, the catheter; and
a middle section between the distal end and the proximal end; the middle section comprising a compressible part that can be clamped to occlude the catheter or a valve that can be closed to occlude the catheter;
wherein the catheter is dimensioned and configured to be inserted through a channel of a bronchoscope.

The catheter can be hollow to allow the flow of air between the detachable adaptor at the proximal end of the catheter and the balloon at the distal end, in order to inflate and deflate the balloon. The catheter can comprise a separate air channel along the catheter to connect the balloon to the proximal end via the detachable adaptor at the proximal end.

Preferably, the catheter has a tip that is soft in order not to injure the airways, and a shaft made of a semi rigid plastic in order to make it both flexible and steerable. Preferably, the tip can be steered within the airway using a force applied to the proximal end of the catheter. The catheter can be steerable from outside the patient, even when the catheter is outside of the bronchoscope channel. This is clinically important, because the patient has respiratory movement, and may cough, and any of these can dislodge the catheter tip from the site of bleeding. So if the location of the tip needs to be adjusted; it can be done by applying maneuvers at the proximal end of the catheter, while guided by the bronchoscope camera.

The catheter can be radiolucent, allowing the catheter to be tracked and seen in simple x-ray or fluoroscopy. This is very helpful to the clinician and allows the clinician to make sure the catheter remains in its desired place, especially when the patient moves, or coughs, or if the equipment has moved.

The compressible part of the catheter can be made of rubber. The catheter can comprise a detachable clamp that can be applied to the compressible part of the catheter to occlude the catheter and permit the balloon to remain inflated when the detachable adaptor is removed from the proximal end of the catheter. The rubber part is designed to prevent puncture to the catheter when a clamp is used to occlude the catheter.

The adaptor at the proximal end of the catheter is able to be detached from the catheter to allow the bronchoscope to slide over the catheter. After that, the adaptor can be connected to the catheter and be used to inflate or deflate the balloon. This design helps to keep the catheter in place and adjust its position and the size of the inflated balloon without the need to replace the catheter. This elimination of the need for replacement of the catheter is especially important in an emergency situation of pulmonary hemorrhage, where one is dealing with a life-threatening situation where every second matters. The detachable adaptor can comprise a two-way stop cock and/or a switching lock to keep air trapped in the balloon. The detachable adaptor can be configured to connect to a syringe that is used to push air into the catheter or into the air channel to inflate the balloon.

The catheter is designed to be used in different airways with different diameter size. It can be made in a spectrum of different diameters and balloon sizes for ease of use and safety. While the diameter of the catheter can range, for example, from 1 mm to 2.5 mm, the outer diameter of the inflated balloon can range, for example, from 5 mm to 20 mm. The diameter of the catheter is small enough that it can be used and positioned inside or alongside of the bronchoscope for any predicted possibility of bleeding, especially during advanced bronchoscopy, cryo-biopsy, or trans-bronchial biopsy of high risk patients such as patients with structural airway disease, such as bronchiectasis.

The catheter has a length that is at least twice the length of the channel of the bronchoscope. This allows the bronchoscope to slide over the catheter and be removed from the airway while the balloon is kept inflated and positioned at a bleeding site.

The invention provides a method of controlling bleeding in the airway, the method comprising
inserting any of the catheters disclosed herein with a deflated balloon into the airway adjacent to a site of bleeding, wherein the catheter is inserted into the airway through a channel of a bronchoscope;
inflating the balloon to apply pressure to the site of bleeding to thereby control bleeding in the airway;
partially withdrawing the bronchoscope by sliding the bronchoscope over the catheter, keeping the catheter in place and the balloon inflated;
when the bronchoscope is withdrawn past the compressible part of the catheter or past the valve in the middle part of the catheter, either clamping the compressible part of the catheter or closing the valve to occlude the catheter, thereby keeping the balloon inflated;
detaching the adaptor from the proximal end of the catheter, and fully withdrawing the bronchoscope past the proximal end of the catheter; and, optionally,
reinserting the bronchoscope into the airway.

In one embodiment, the method comprises passing the balloon catheter via the working channel of a bronchoscope while the balloon is deflated, and compressing the bleeding site or blocking the bleeding airways by inflating the balloon under direct visualization without the need to remove the bronchoscope from the bleeding site. The balloon can be inflated with air with the help of a syringe, and the adaptor attached to the proximal end of the catheter channel. Then the adaptor will then be locked. After the balloon is inflated, the bronchoscope can be removed from the site without the need to move the catheter or deflate the balloon. First, the bronchoscope can be slid over the catheter toward the proximal end of the catheter while applying forward pressure over the catheter to keep the distal end of the catheter in place. When the bronchoscope passes the compressible part of the catheter, the clamp can be used to squeeze the compressible part and block the catheter. Then the adaptor at the proximal end of the catheter can be detached. This allows the adaptor to be detached without deflating the balloon. Then the bronchoscope can be removed from the proximal part of the catheter by sliding over the rest of catheter. The bronchoscope can then be immediately reinserted into the airway to check the status of the balloon and airway bleeding. If the balloon position needs to be adjusted, it can be done with the help of the bronchoscope.

The invention also provides a method of controlling bleeding in the airway, the method comprising
inserting any of the catheters disclosed herein with a deflated balloon into the airway adjacent to a site of bleeding, wherein the catheter is inserted into the airway through a channel of a bronchoscope;
detaching the adaptor from the proximal end of the catheter;
removing the bronchoscope by sliding the bronchoscope over the catheter, keeping the catheter in place;
reattaching the adapter to the proximal end of the bronchoscope;
inflating the balloon to apply pressure to the site of bleeding to thereby control bleeding in the airway and, optionally,
reinserting the bronchoscope into the airway.

The methods can further comprise deflating the balloon, repositioning the catheter within the airway, and reinflating the balloon.

The invention further provides a method of retracting a foreign body from the airway, the method comprising
inserting any of the catheters disclosed herein with a deflated balloon into the airway adjacent to the foreign body, wherein the catheter is inserted into the airway through a channel of a bronchoscope;
detaching the adaptor from the proximal end of the catheter;
removing the bronchoscope by sliding the bronchoscope over the catheter, keeping the catheter in place;
reinserting the bronchoscope into the airway to visually guide the distal end of the catheter past the foreign body; reattaching the adapter to the proximal end of the bronchoscope;
inflating the balloon; and either
using the inflated balloon to retract the foreign body as the catheter with the inflated balloon is withdrawn from the airway, or
using the inflated balloon to prevent movement of the foreign body within the airway while another tool is inserted into the airway to grab and retract the foreign body.

This invention provides balloon catheters that can be inserted via the working channel of the bronchoscope in a timely manner. The bronchoscope does not need to be removed during the insertion of the catheter, so the operator can continue to use other techniques to stop bleeding, while preparing the balloon insertion. The balloon catheters of the present invention are able to be detached and separated from the bronchoscope while the balloon remains inflated and positioned at the airway bleeding site. This is a great advantage, since it allows the operator to use the bronchoscope for other steps while the bleeding site is covered with the balloon. The operator can then check other neighborhood airways and clean them from any possible spilled blood and also remove any clots that may jeopardize the ventilation and oxygenation of the patient. At the same time, the bronchoscope can examine the position of the balloon and make sure it stays in the correct place. Other interventions can be performed, including using cold saline, and securing the airway if the patient has not been intubated before. This invention can be used prophylactically in cases where bleeding during trans-bronchial biopsy is highly expected, meaning the catheter can be positioned close to the site of biopsy, and if major bleeding happens, the balloon can be quickly inflated. If the balloon needs to remain in the airway for a longer period of time, and patient needs to be transferred to any other units or for a surgery, it will be easier to transfer the patient, since the bronchoscope can be removed from the patient, while the catheter remains in place.

1. A catheter for insertion into an airway of a patient through a channel of a bronchoscope, the catheter comprising
a distal end, which is inserted into a patient, the distal end comprising a balloon that can be inflated and deflated;
a proximal end, which is the end closest to a physician or operater when the distal end is inserted into the patient, the proximal end comprising an adaptor used to inflate and deflate the balloon, wherein the adaptor is detachable from, and reattachable to, the catheter; and
a middle section between the distal end and the proximal end; the middle section comprising a compressible part that can be clamped to occlude the catheter or a valve that can be closed to occlude the catheter;
wherein the catheter is dimensioned and configured to be inserted through a channel of a bronchoscope.

2. The catheter of claim 1, wherein the catheter comprises an air channel along the catheter to connect the balloon to the proximal end via the detachable adaptor at the proximal end.

3. The catheter of claim 1, wherein the distal end of the catheter has a soft tip and the catheter has a shaft made of a semi rigid plastic.

4. The catheter of claim 3, wherein the tip can be steered within the airway using a force applied to the proximal end of the catheter.

5. The catheter of claim 1, wherein the catheter is radiolucent.

6. The catheter of claim 1, wherein the compressible part of the catheter is made of rubber.

7. The catheter of claim 1, wherein the catheter comprises a detachable clamp that can be applied to the compressible part of the catheter.

8. The catheter of claim 1, wherein the detachable adaptor comprises a two-way stop cock.

9. The catheter of claim 1, wherein the detachable adaptor is configured to connect to a syringe that is used to push air into the catheter or into the air channel to inflate the balloon.

10. The catheter of claim 1, wherein the detachable adaptor has a switching lock to keep the air trapped in the balloon.

11. The catheter of claim 1, wherein the catheter has a diameter of 1 mm to 2.5 mm.

12. The catheter of claim 1, wherein the balloon when inflated has a diameter of 5 mm to 20 mm.

13. The catheter of claim 1, wherein the catheter has a length that is at least twice the length of the channel of the bronchoscope.

14. A method of controlling bleeding in the airway, the method comprising
inserting the catheter of claim 1 with a deflated balloon into the airway adjacent to a site of bleeding, wherein the catheter is inserted into the airway through a channel of a bronchoscope;
inflating the balloon to apply pressure to the site of bleeding to thereby control bleeding in the airway;
partially withdrawing the bronchoscope by sliding the bronchoscope over the catheter, keeping the catheter in place and the balloon inflated;
when the bronchoscope is withdrawn past the compressible part of the catheter or past the valve in the middle part of the catheter, either clamping the compressible part of the catheter or closing the valve to occlude the catheter, thereby keeping the balloon inflated;
detaching the adaptor from the proximal end of the catheter, and fully withdrawing the bronchoscope past the proximal end of the catheter; and, optionally,
reinserting the bronchoscope into the airway.

15. A method of controlling bleeding in the airway, the method comprising
inserting the catheter of claim 1 with a deflated balloon into the airway adjacent to a site of bleeding, wherein the catheter is inserted into the airway through a channel of a bronchoscope;
detaching the adaptor from the proximal end of the catheter;
removing the bronchoscope by sliding the bronchoscope over the catheter, keeping the catheter in place;
reattaching the adapter to the proximal end of the bronchoscope;
inflating the balloon to apply pressure to the site of bleeding to thereby control bleeding in the airway and, optionally,
reinserting the bronchoscope into the airway.

16. The method of claim 14, which further comprises deflating the balloon, repositioning the catheter within the airway, and reinflating the balloon.

17. A method of retracting a foreign body from the airway, the method comprising
inserting the catheter of claim 1 with a deflated balloon into the airway adjacent to the foreign body, wherein the catheter is inserted into the airway through a channel of a bronchoscope;
detaching the adaptor from the proximal end of the catheter;
removing the bronchoscope by sliding the bronchoscope over the catheter, keeping the catheter in place;
reinserting the bronchoscope into the airway to visually guide the distal end of the catheter past the foreign body;
reattaching the adapter to the proximal end of the bronchoscope;
inflating the balloon; and either
using the inflated balloon to retract the foreign body as the catheter with the inflated balloon is withdrawn from the airway, or
using the inflated balloon to prevent movement of the foreign body within the airway while a tool is inserted into the airway to grab and retract the foreign body.

18. The method of claim 15, which further comprises deflating the balloon, repositioning the catheter within the airway, and reinflating the balloon.
